(19) United States
(12) Patent Application Publication
(10) Pub. No.: US 2003/0219835 A1
(43) Pub. Date: Nov. 27, 2003

(54) CD16b as a marker for the diagnosis of endometriosis

(75) Inventors: Diane Gosselin, Pointe Calumet (CA); Daniele Gagne, Iberville (CA); Martin Page, Sorel-Tracy (CA); Michele Rivard, Ile des Soeurs (CA); Christele Platon, Verdun (CA); Manon Lepine, Laval (CA); Kamran Shazand, Verdun (CA); Patrice Hugo, Montreal (CA)

Correspondence Address: CROWELL & MORING LLP, INTELLECTUAL PROPERTY GROUP, P.O. BOX 14300, WASHINGTON, DC, 20044-4300, US

(73) Assignee: Metriogene Biosciences Inc., Montreal (CA)

(21) Appl. No.: 10/364,651

(22) Filed: Feb. 12, 2003

Related U.S. Application Data
(60) Provisional application No. 60/355,818, filed on Feb. 13, 2002.

(51) Int. Cl. G01N 33/53; G01N 33/567

(52) U.S. Cl. 435/7.2

(57) ABSTRACT

The invention relates to methods and a kit for the diagnosis of endometriosis. More particularly, the invention relates to the measurement of endometrial leukocytes bearing CD16b and, optionally, other leukocyte or serous markers, for determining likelihood of suffering from endometriosis in a female subject.

### FIELD OF THE INVENTION

The invention relates to methods and a kit for the diagnosis of endometriosis. More particularly, the invention relates to the measurement of endometrial leukocytes defined by the expression of CD16b and, optionally, other leukocyte or serous markers, for determining likelihood of suffering from endometriosis in a female subject.

### BACKGROUND OF THE INVENTION

Endometriosis is one of the most common gynecological disorders, affecting up to 10-15% of women of reproductive age. It is mainly associated with severe pelvic pain and/or infertility, but also with dysmenorrhea, dyspareunia, and several other symptoms such as intraperitoneal bleeding, back pain, constipation and/or diarrhea. Endometriosis is characterized by the implantation and growth of endometrial cells (which normally constitute the lining of the uterus) in extra-uterine sites, most frequently in the peritoneal cavity.

At present, direct visualization of the endometriotic lesions under surgical procedures (laparoscopy or laparotomy) is the only reliable method to diagnose endometriosis. However, this method is highly invasive (i.e. surgery under general anesthesia) and costly. The period of time between the onset of symptoms and disease diagnosis can be as long as 8 to 12 years. Ideally, the prospect to diagnose endometriosis more easily, rapidly, and as early as possible during the course of the disease would definitely reduce the number of years during which patients endure pain, infertility or other symptoms.

Based on this perspective, several investigators have sought to identify biological markers (proteic and genetic) that could efficiently be used as predictive tools for endometriosis. For instance, in International PCT application PCT/CA00/00060 filed on Jan. 24, 2000 and published under No. WO 00/43789, the present Applicant describes a series of blood and endometrial leukocyte markers that may be useful for determining the likelihood of suffering from endometriosis. However, prior to the present invention, there was no evidence of an association of CD16b with endometriosis and such a use was not suggested in the above-mentioned PCT application nor in any other document. Overall, no one has ever described any method for determining in females the likelihood of suffering from endometriosis by measuring endometrial leukocytes bearing CD16b, nor any method involving the CD16b for efficiently identifying females suffering from endometriosis.

There is therefore a need for an alternative approach to laparoscopy or laparotomy to determine the likelihood of females to suffer from endometriosis and to diagnose endometriosis. The numerous limitations of these methods establish the need for a less invasive, and more rapid diagnostic test for endometriosis based on the detection of biological markers. More particularly, it would be highly desirable to be provided with methods wherein quantitative levels of CD16b expressing endometrial leukocytes are measured for the diagnosis of endometriosis.

The present invention fulfils these needs and also other needs that will be apparent to those skilled in the art upon reading the following specification.

### SUMMARY OF THE INVENTION

One aim of the present invention is to provide methods and a kit for determining the likelihood of female subjects of suffering from endometriosis.

The present inventors have found that levels of leukocytes defined by the expression of CD16b at their surface are significantly modulated in the endometrium of patients with endometriosis compared to normal controls.

In accordance with an aspect of the present invention, there is provided a method for determining likelihood of endometriosis in a female subject, comprising the steps of:

a) obtaining from said female a sample of uterine endometrial tissues; and

b) measuring in said sample a quantitative level of a population of CD16b+ endometrial leukocytes,

wherein the quantitative level measured in step b) is indicative of an increased likelihood of endometriosis in said female subject as compared to an endometriosis-free female subject.

In accordance with another aspect of the invention, it is provided a method for determining likelihood of endometriosis in a female subject, comprising the steps of:

a) evaluating in said female subject a quantitative level of a population of CD16b+ endometrial leukocytes and a quantitative level of at least one further population of endometrial leukocytes, among total endometrial leukocytes;

b) establishing a cutoff value for each quantitative level evaluated in step a);

c) comparing each of said quantitative levels evaluated in step a) with the cutoff value defined in step b) and assigning a first value if said quantitative level meets a condition established by the cutoff value, or assigning a second value different from the first value if said quantitative level does not meet the condition established by the cutoff value;

d) adding up the values assigned in step c) to obtain a score;

e) defining a threshold value for the score obtained in step d); and

f) comparing the score obtained in step d) to the threshold value defined in step e);

wherein when said score is higher than said threshold value, there is an indication of an increased or a likelihood of endometriosis in said female subject as compared to an endometriosis-free female subject.

In accordance with further aspect of the present invention, it is provided a method for determining likelihood of suffering from endometriosis in a female subject, the method comprising the steps of:

a) obtaining from said female a sample of uterine endometrial tissues;

b) determining in said sample a quantitative level of CD16b+, CD3−CD45RA−, CD3−HLADR−, CD3+, CD56−CD16+, CD3+CD16−, CD3+CD56−, and CD16+ leukocytes;

c) obtaining a blood sample from the female subject;

d) determining in said blood sample a quantitative level of CA-125;

e) evaluating in the female subject a medical condition selected from the group consisting of the number of pregnancies, the length of periods and the day of the menstrual cycle at which the endometrial tissue is sampled;

wherein when combined, the endometrial leukocytes quantitative levels, the CA-125 quantitative level and the medical condition(s) are indicative of a higher or a lower likelihood of endometriosis in the female subject as compared to an endometriosis-free female subject.

Yet, according to another aspect, the present invention provides a diagnostic kit for determining likelihood of endometriosis in a female subject. The kit, comprises at least one binding agent that specifically binds to a CD16b+ leukocyte and a reagent for detecting the binding agent/CD16b+ leukocyte binding complex.

An advantage of the present invention is that it is rapid, less invasive than surgery and significantly less complicated and costly than performing laparoscopy or laparotomy. Moreover, it is possible, according to the present invention, to directly measure, without surgery, likelihood of endometriosis with high levels of sensitivity and specificity. The invention therefore provides a much more accessible test for determining the likelihood of suffering from endometriosis.

Other objects and advantages of the present invention will be apparent upon reading the following non-restrictive description.

### BRIEF DESCRIPTION OF THE DRAWINGS

FIG. 1 is a scheme for the diagnosis of endometriosis resulting from a method according to a preferred embodiment of the invention.

### DETAILED DESCRIPTION OF THE INVENTION

A) Definitions

In order to provide a clearer and more consistent understanding of the specification and the claims, including the scope given herein to such terms, the following definitions are provided:

Endometrial cells: Refer to the cells that are lining the uterus. Normally, endometrial cells are sloughed off during the woman's menstrual period, and afterwards the cellular layer grows back and slowly thickens until the next period. As used herein, “endometrial cells” encompasses only eutopic endometrial cells (i.e. the cells that usually constitute the lining of the uterine cavity) as opposed to those cells outside the uterus that are considered “ectopic”.

Female subject: Refers to human females being in reproductive age. According to the present invention, the female subject would preferably present clinical symptoms of endometriosis such as infertility and pelvic pain.

Leukocyte population: Refers to a subset of leukocytes having a specific characteristic (e.g. a definite surface molecule), among all leukocytes present in a given leukocytes sample.

Likelihood: As used herein in combination with the term “endometriosis”, it more particularly refers to an existing probability of a female subject of actually suffering from endometriosis. It does not refer to a predisposition to suffering in the future from the disease.

Phase of menstrual cycle: Refers to the period of menstrual cycle in which physiological changes occur in females as a result of hormonal influences during the menstrual or ovarian cycle. Briefly, in human females, the menstrual cycle is divided into two phases, namely, the “proliferative phase” (also called the follicular phase, herein referred to as P phase) and the “secretory phase” (also called the luteal phase, herein referred to as S phase). The proliferative phase normally extends from day 0 to day 14 of the menstrual cycle, and the secretory phase normally extends from day 15 to day 28, ovulation occurring on day 14 of a regular menstrual cycle.

Quantitative level: As used herein with the term leukocyte population, it refers to the measure of the proportion of a specific subset of leukocytes with respect to all the leukocytes present in a given endometrial biopsy. Depending on the specific uses, the quantitative level may be very precise or only approximative.

B) General Overview of the Invention

The present invention concerns the early detection, diagnosis and prognosis of endometriosis.

As it will be demonstrated hereinafter in the exemplification section, an extensive study was undertaken by means of flow cytometric analysis, in which the proportion of several endometrial leukocyte subsets was compared in patients with endometriosis (stage I-IV) or without endometriosis. It was found that levels of leukocytes defined by the expression of CD16b at their surface are significantly modulated in the endometrium of patients with endometriosis compared to normal controls.

Therefore the essence of the present invention is the use of the CD16b positive endometrial leukocyte subpopulation, either alone or in combination with other endometrial leukocyte subpopulations, as markers for detecting females with high likelihood of suffering from endometriosis. Preferably, leukocytes expressing CD16b (also called FcγRIIIb) are typically, but not exclusively granulocytes.

Moreover, CA-125 serum level was shown to be of significant value when used in combination with endometrial leukocyte subsets and CD16b+ endometrial cells. Finally, risk factors for endometriosis identified among personal information and menstrual characteristics were also shown to be of significant value when used in combination with quantitative levels of endometrial leukocyte subsets and CA-125 serum level in a diagnostic test for endometriosis.

i) Methods of the Invention

In accordance with the present invention, there is provided methods for determining the likelihood of female subjects of suffering from endometriosis and a reliable diagnostic test for endometriosis that is less invasive and less costly than the actual surgical procedure accepted as the gold standard.

According to a first embodiment of the invention, the method comprises the steps of:

a) obtaining a sample of uterine endometrial tissue from a female subject, preferably during the secretory phase of the menstrual cycle; and

b) determining in the sample the quantitative level of a population of CD16b+ endometrial leukocytes.

In a preferred embodiment, the method further comprises a step c) of comparing the quantitative level to a predetermined cut-off value, and wherein an increased quantitative level of the population of CD16b+ leukocytes as compared to the cut-off value is indicative of an increased likelihood of suffering from endometriosis in the female subject as compared to an endometriosis-free female subject. Preferably, the quantitative level of said population of CD16b+ endometrial leukocytes corresponds to a proportion of a population of eutopic CD16b+ endometrial leukocytes among total endometrial leukocytes of said female subject. Moreover, the proportion of leukocytes is preferably determined by using an antibody specific for the population of CD16b+ leukocytes.

Advantageously, in step c), the cutoff value is calculated and obtained by using the following steps:

determining a first quantitative level for the population of CD16b+ leukocytes in a positive reference group of female subjects suffering from endometriosis;

determining a second quantitative level for the population of CD16b+ leukocytes in a negative reference group of endometriosis-free female subjects; and

calculating the cutoff value with the first and second quantitative levels.

According to a preferred embodiment, the method further comprises the step of measuring the quantitative level of at least one further population of endometrial or blood leukocytes. The Applicant's International PCT application No. WO 00/43789 (incorporated herein by reference) gives a list of blood and endometrial leukocyte markers that may be useful according to the present invention. More preferably, in order to increase the diagnostic performance of the method, the quantitative level of at least one further population of endometrial leukocytes is evaluated, these being selected from the group consisting of CD3−HLADR−, CD3+, CD56−CD16+, CD3+CD16−, CD3+CD56−, CD3−CD45RA− and CD16+.

More preferably, in order to further increase the diagnostic performance of the method, the method of the invention also comprises the steps of obtaining a blood sample from the subject female and measuring the level of CA-125 in the serum.

According to another preferred embodiment, acquisition of some clinical information also increases the diagnostic performance of the method. The clinical information that may be acquired includes the number of pregnancies, the length of the menstruation, and the date in the menstrual cycle at which the endometrial tissue is taken.

According to a second embodiment of the invention, it is provided a method for determining likelihood of suffering from endometriosis in a female subject. Such a method comprises the steps of:

a) evaluating in said female subject a quantitative level of a population of CD16b+ endometrial leukocytes and a quantitative level of at least one further population of endometrial leukocytes, among total endometrial leukocytes;

b) establishing a cutoff value for each quantitative level evaluated in step a);

c) comparing each of said quantitative levels evaluated in step a) with the cutoff value defined in step b) and assigning a first value if said quantitative level meets a condition established by the cutoff value, or assigning a second value different from the first value if said quantitative level does not meet the condition established by the cutoff value;

d) adding up the values assigned in step c) to obtain a score;

e) defining a threshold value for the score obtained in step d); and

f) comparing the score obtained in step d) to the threshold value defined in step e);

wherein when said score is higher than said threshold value, there is an indication of an increased or a likelihood of endometriosis in said female subject as compared to an endometriosis-free female subject.

Advantageously, the cutoff value mentioned in step b) is calculated and obtained by:

determining a first reference quantitative level for the population of CD16b+ endometrial leukocytes and for the at least one specific population of leukocytes in a positive reference group of female subjects suffering from endometriosis;

determining a second reference quantitative level for the population of CD16b+ endometrial leukocytes and for the at least one specific population of leukocytes in a negative reference group of endometriosis-free female subjects; and

calculating said cutoff value with the first and second reference quantitative levels

Preferably, in step e) of the method of the second embodiment, the threshold value is calculated and obtained by using the steps of:

applying steps a) to d) to a positive reference group of female subjects suffering from endometriosis to obtain a first reference score;

applying steps a) to d) to a negative reference group of endometriosis-free female subjects to obtain a second reference score; and

calculating said threshold value with said first and second reference score.

According to a third embodiment of the invention, it is provided a method for determining likelihood of endometriosis in a female subject. Such a method comprises the steps of:

a) evaluating in said female subject a quantitative level of a population of CD16b+ endometrial leukocytes and a quantitative level of at least one further population of endometrial leukocytes, among total endometrial leukocytes;

b) establishing a cutoff value for each quantitative level evaluated in step a);

c) comparing each of said quantitative levels evaluated in step a) with the cutoff value defined in step b) and assigning a first value if said quantitative level meets a condition established by the cutoff value, or assigning a second value different from the first value if said quantitative level does not meet the condition established by the cutoff value; and

d) processing the first or second value of step c) in a logistic regression model in order to obtain a score, said score determining the likelihood of endometriosis in said female subject.

According to a fourth embodiment, the present invention provides a method for determining likelihood of suffering from endometriosis in a female subject. Such a method comprises the steps of: obtaining a sample of uterine endometrial tissues (preferably taken during the secretory phase of the menstrual cycle) from the female subject and determining in the sample a quantitative level of CD16b+, CD3−CD45RA−, CD3−HLADR−, CD3+, CD56−CD16+, CD3+CD16−, CD3+CD56−, and CD16+ leukocytes. The method also comprises a step of obtaining a blood sample from the female subject and determining in the serum a quantitative level of CA-125. Finally, the method has a step of evaluating in the female subject a medical condition selected from the group consisting of the number of pregnancies, the length of periods and the day of the menstrual cycle at which the endometrial tissue is sampled. Thus, when combined, these endometrial leukocytes quantitative levels, the CA-125 quantitative level and the medical condition(s) are indicative of a higher or a lower likelihood of endometriosis in the female subject as compared to an endometriosis-free female subject. As can be appreciated, the method according to the fourth embodiment is an improved method for determining likelihood of suffering from endometriosis in a female subject since level of CA-125 in serum is advantageously measured. Indeed, elevated levels of CA-125 in serum menstrual effluent and peritoneal fluid of patients has been associated with endometriosis (Mol et al., (1998) Fertility and Sterility 70 :1101-1108). The improved method according to the present invention is characterized in that CA-125 measurement in serum is combined with the measurement of at least one endometrial leukocyte population. As it will be shown hereinafter, it has been found that the diagnostic value of CA-125 is greatly increased by combining the measured levels of this marker to endometrial leukocyte levels measurement.

For determining the quantitative level of the selected leukocytes population(s) in the endometrium, many methods and tools may be used. Since the leukocyte markers of the invention are surface molecules, antibodies are a preferred tool. Therefore, the method of the invention preferably comprises the use of labeled monoclonal or polyclonal antibodies specific against a definite surface molecule (e.g. CD16b antigen) to identify the leukocyte cell population (e.g. CD16b positive cells), more preferably by flow cytometry analysis. Examples of other suitable means include immunofluorescence, immunochemistry, ELISA, RIA, and Western blot.

A person skilled in the art will understand that the invention is not restricted to a definite method or tool since many other methods and tools could also be used for identifying the same leukocyte population(s). A not exclusive list of examples includes: measurement of the expression of other cell surface or intracellular molecules; measurement of the secretion of specific enzymes, cytokines, growth factors, adhesion molecules, inflammatory mediators and the like; measurement of specific cell function(s) (e.g. capacity to lyse a specific population of cells); morphology analysis; measurement of the capacity to adhere to plastic; measurement of the phagocytosis capacity; measurement of the capacity to be activated by specific cytokines or molecules, etc.

As mentioned previously, it is also preferable according to the present invention to compare the leukocyte quantitative level to a cut-off value in order to obtain the best discrimination between females with endometriosis and control. Table 2 in the exemplification section provides an example of a preferred cut-off value for CD16b. Since it is well known in the art how to calculate such a cut-off value, and that the best cut-off may vary according to many factors such as the desired sensitivity and specificity of the marker, the calculation method will not be described further.

When using a plurality of parameters (e.g. a combination of CA-125 serum level, and/or endometrial leukocyte marker(s), a given medical condition), it is also advantageous to use a predictive model to obtain the best discrimination between patients with endometriosis and controls. Example 1 hereinafter gives a specific example of a logistic regression model for calculating the likelihood of a female of having endometriosis. It is to be understood that many other statistical models and methods could also be used for evaluating the probability of a female of suffering from endometriosis. These are believed to be within the skill of persons to the invention pertains.

ii) Kit

According to a fifth embodiment, the present invention relates to a diagnostic kit for determining likelihood of endometriosis in a female subject suspected to suffer from the disease. The kit of the invention comprises at least one binding agent for binding to a CD16b leukocyte; and a reagent for detecting the binding agent/CD16b leukocyte binding complex. Preferably, the kit further comprises at least one element selected from the group consisting of: a support for the binding agent(s), mixing tubes, buffers, enzymes, and instructions for using the kit.

Preferably, the binding agent for binding the CD16b is a labeled monoclonal or polyclonal antibody. Most preferred antibodies include mouse anti-CD16b monoclonal antibodies labeled with FITC such as those sold by Beckman Coulter.

Preferably, the kit comprises at least another binding agent that specifically binds to another population of leukocytes such as one selected from the group consisting of CD3−HLADR−, CD3+, CD56−CD16+, CD3+CD16−, CD3+CD56−, CD3−CD45RA− and CD16+. Yet, the present invention advantageously comprises at least one FURTHER binding agent that specifically binds to CA-125.

Advantageously, the kit of the present invention may also comprises a software which would allow a user to enter numerous parameters such as information on the patient (name, medical condition, etc.) and the results of the leukocytes measurement(s). The software would then process the entered data and calculate the likelihood for the patient to have endometriosis.

### EXAMPLE

The following example illustrates the wide range of potential applications of the present invention and is not intended to limit its scope. Modifications and variations can be made therein without departing from the spirit and scope of the invention. Although any methods and materials similar or equivalent to those described herein can be used in the practice for testing the present invention, the preferred methods and materials are described.

### Example 1

### CD16b as a Marker of Endometriosis

1) Methods

Study Patients and Samples

Patients were recruited among women who were scheduled to undergo laparoscopy or laparotomy. Gynecologists collaborating in the study were trained surgeons experienced with the management of endometriosis. To be admitted into the study, patients had to be of premenopausal age, not currently menstruating, have regular menstrual cycles (between 21 and 35 days), have no acute salpingitis, have not been pregnant for the last three months; have not been under hormonal treatment for the last three months, nor using intra-uterine device for the last three months.

Uterine endometrial tissues were obtained from 368 patients undergoing laparoscopy or laparotomy. The group of cases was formed of 173 patients with endometriosis stage I-IV confirmed by laparoscopy or laparotomy and the control group consisted of 195 patients who underwent surgery for several different indications (e.g. tubal ligation, diagnostic laparoscopy or hysterectomy) and had no clinical evidence of endometriosis.

Endometrial biopsies were taken with a Pipet Curette™ (Milex) (approximately 0.5 g of tissue). All samples were harvested in the secretory phase (day 15-28) of the menstrual cycle as confirmed by histological evaluation. The samples were collected into sterile RPMI-1640 medium (Gibco) supplemented with 2% heat-inactivated fetal calf serum (Bio-Media) and 1% penicillin-streptomycin and kept at 4° C. until cell isolation. Blood samples were collected in tubes containing no additive (Vacutainer™, Becton Dickinson) and kept at 20° C.

Stromal Cell Preparation from Endometrial Samples

Endometrial tissue samples were mechanically disrupted with a Pyrex™ glass Broeck tissue grinder (Fisher) to obtain a single cell suspension. Stromal cell fraction was isolated by filtration through a 250 μm stainless steel sieve (Millipore) to retain the glandular fraction and was washed once with 10 ml phosphate buffered saline (PBS) (Sigma).

Preparation of Serum from Peripheral Blood

Blood samples were allowed to clot for at least 1 hour and centrifuged at 1100 rpm for 10 minutes. Supernatant was collected and stored at −80C. until CA-125 level determination.

Endometrial Leukocyte Surface Antigen Staining

Endometrial stromal cells were distributed in 5 ml tubes (1 to 1.5×10⁶ cells/tube) and incubated in the presence of 0.1 μg of human γ-globulin for 5 minutes at room temperature. The cells were then incubated 30 minutes in the dark at room temperature with mouse monoclonal anti-human antibodies (MAbs) listed in Table 1 in a total volume of 100 μl. The cell samples were stained with mouse anti-human CD45 MAbs conjugated to peridinin chlorophyl protein (PerCP) and with up to 2 different mouse MAbs labeled with distinct fluorochromes (fluorescein isothiocyanate—FITC—, phycoerythrin—PE or with phycoerythrin-texas red—ECD—) directed toward cell surface markers for specific cell populations.

Cell samples were then incubated with a red blood cell lysing solution, (FACS™ Lysing Solution, Becton Dickinson) for 10 minutes at room temperature in the dark and washed with 2 ml of PBS washing buffer. Endometrial cells were fixed in 1% paraformaldehyde (diluted in PBS) at a concentration of 1.5×10⁶ cells/ml and kept at 4° C. in the dark until the immunofluorescence reactivity was determined by flow cytometry.

TABLE 1
List of monoclonal antibodies that were used to define endometrial
leukocyte subsets under investigation.

| Endometrial leukocyte | Antibodies used to define specific |
|---|---|
| subsets detected | leukocyte subsets¹ |
| CD16b+ | Anti-CD16b labeled with FITC; |
| | Anti-CD45 labeled with PercP |
| CD16+ | Anti-CD16 labeled with FITC; |
| | Anti-CD45 labeled with PercP |
| CD3+ | Anti-CD3 labeled with ECD; |
| | Anti-CD45 labeled with PercP |
| CD3−HLADR− | Anti-CD3 labeled with ECD; |
| | Anti-HLADR labeled with FITC; |
| | Anti-CD45 labeled with PercP |
| CD3−CD45RA− | Anti-CD3 labeled with ECD; |
| | Anti-CD45RA labeled with FITC; |
| | Anti-CD45 labeled with PercP |
| CD56−CD16+ | Anti-CD56 labeled with PE; |
| | Anti-CD16 labeled FITC; |
| | Anti-CD45 labeled with PercP |
| CD3+CD16− | Anti-CD3 labeled with ECD; |
| | Anti-CD16 labeled FITC; |
| | Anti-CD45 labeled with PercP |
| CD3+CD56− | Anti-CD3 labeled with ECD; |
| | Anti-CD56 labeled with PE; |
| | Anti-CD45 labeled with PercP |

¹Fluorochromes are abbreviated as followed: ECD: phycoerythrin-Texas Red; PerCP: peridinin chlorophyl protein; PE: phycoerythrin; FITC: fluorescein isothiocyanate.

Flow Cytometry Analysis

The immunofluorescence reactivity was carried out on a Coulter EPICS XL™ flow cytometer (Coulter Corporation, Hialeah, Fla.) equipped with an argon laser operating at 488 nm, 15 mW and detectors at 525, 575, 610, and 675 nm. Calibration of the flow cytometer parameters for forward scatter, side scatter and fluorescence were the same for all the samples. Cells expressing CD45 pan leukocyte antigen were gated using the Coulter system II software. The percentage of cells bearing the surface markers of interest (Table1) was evaluated within the CD45 positive population of leukocytes only. A minimum of 6000 CD45+ cells were analyzed for each sample.

Measurement of CA-125 Levels in Serum Samples

The concentration of CA-125 in serum samples was determined by means of a one step-sandwich radioimmunoassay (RIA) (Fujirebio America Inc.). Briefly, 100 μl of undiluted serum samples were incubated overnight in duplicate with polystyrene beads coated with anti CA-125 mAbs (capture antibody) and with the tracer antibody, which consists of ¹²⁵I-labeled anti CA-125 mAbs (with different specificity than capture antibodies). During this incubation, molecules containing CA-25 determinant in the serum formed complexes with the monoclonal antibodies and beads. Unbound molecules in the serum were removed by washing the beads. The bound radioactivity is proportional to the CA-125 concentration in serum samples. CA-125 serum levels were determined by comparison to a standard curve. CA-125 serum level was expressed in U/ml serum according to the manufacturer's instructions. A total of 2 controls were included in each individual experiment. Intra-assay and inter-assay variations of less than 10% were accepted for this study.

Combination of Several Markers in a Diagnostic Test for Endometriosis

The method used to combine endometrial leukocyte markers, CA-125 serum levels and risk factors for endometriosis was as follows. A cut-off point is established for the quantitative level of each leukocyte markers, CA-125 serum levels and risk factors in order to obtain the best discrimination between patients with endometriosis and controls. The quantitative level obtained for each marker is compared to the cut-off point. If the quantitative level measured for a particular marker (endometrial leukocyte subset, CA-125 serum level or risk factors) fulfills the criteria established by the cut-off point, a score of 1 is given, whereas a score of 0 is given when the quantitative level measured for a particular marker does not fulfill the criteria established by the cut-off point. The probability of suffering from endometriosis is calculated by including the score calculated for each marker in the following logistic regression equation:

$$\underline{P ( r ) =} \frac{{ⅇ}^{c + {B1}^{*} ( marker1 ) + B2 ( marker2 ) + … Bk ( markerk )}}{1 + {ⅇ}^{c + {B1}^{*} ( marker1 ) + B2 ( marker2 ) + … Bk ( markerk )}}$$

Where:

P(r)=probability of having endometriosis;

c=constant established for a particular combination;

B=coefficient of regression; and

k=total number of markers in the combination;

The probability of having endometriosis (P(r)) is then compared to a threshold value that provides the best discriminative value. A positive diagnosis of endometriosis is given when the P(r) value exceeds the threshold value. Alternatively, a negative diagnosis of endometriosis is given when the P(r) value is lower than the threshold value.

2) Results

The quantitative level of endometrial leukocyte subset, defined by the expression of CD16b+ surface molecule, was shown to be significantly modulated in patients with endometriosis compared to controls. This was evaluated by a comparison of mean proportion of CD16b expressing cells in endometrium of patients with endometriosis and normal controls (Table 2). The diagnostic value of this endometrial leukocyte subset was also assessed by measuring the area under the ROC curve, an indication of the discriminative value of the marker. The ROC curve allowed the determination of the cut-off point that best discriminate between patients with endometriosis (stage I-IV) and normal controls (Table 2). In an attempt to use this difference for identifying patients with endometriosis, a positive result was given when the proportion measured for CD16b+ endometrial leukocyte subset fulfilled the condition established by the cutoff point (>13.5), whereas a negative result was given when the proportion of CD16b+ endometrial leukocytes did not fulfill the condition of the cut-off point. The level of specificity (% of negative results among controls) and the level of sensitivity (% of positive results among patients with endometriosis) were calculated according to the above-mentioned procedure (Table 2). Finally, the odds ratio calculated with the pre-established cut-off point indicates that a modulation of the proportion of CD16b+ endometrial leukocyte subset is clearly associated with an increased risk of suffering from endometriosis. Overall, results obtained with the comparison of means, ROC curve analysis as well as the levels of specificity/sensitivity and the odds ratio indicate that modulation of the proportion of CD16b+ endometrial leukocyte subset can be used for identifying women with high likelihood of suffering from endometriosis (Table 2).

TABLE 2
Diagnostic value of endometrial leukocyte subsets defined by expression of CD16b molecule

| | Mean % leukocyte | | | ROC curve | | | | | |
|---|---|---|---|---|---|---|---|---|---|
| Leukocyte | subsets ± S.D. | | P | Area | P | Cut-off | Specificity | Sensitivity | Odds ratio |
| subsets | Controls | Endo I-IV | value | under curve | value | point | (%) | (%) | (95% CI) |
| CD16b+ | 24.6 ± 13.5 | 28.8 ± 15.0 | 0.017 | 0.583 | 0.023 | >13.5 | 27% | 85% | 2.0 |
| | | | | | | | | | (1.1-3.7) |

The overall performance of CD16b+ endometrial leukocyte subset as a diagnostic marker was significantly improved when this marker was used in combination with other endometrial leukocyte markers such as CD3−HLADR−, CD3+, CD3−CD45RA−, CD56−CD16+, CD3+CD16−, CD3+CD56− and CD16+ as well as CA-125 serum level and risk factors for endometriosis such as length of periods and number of pregnancies.

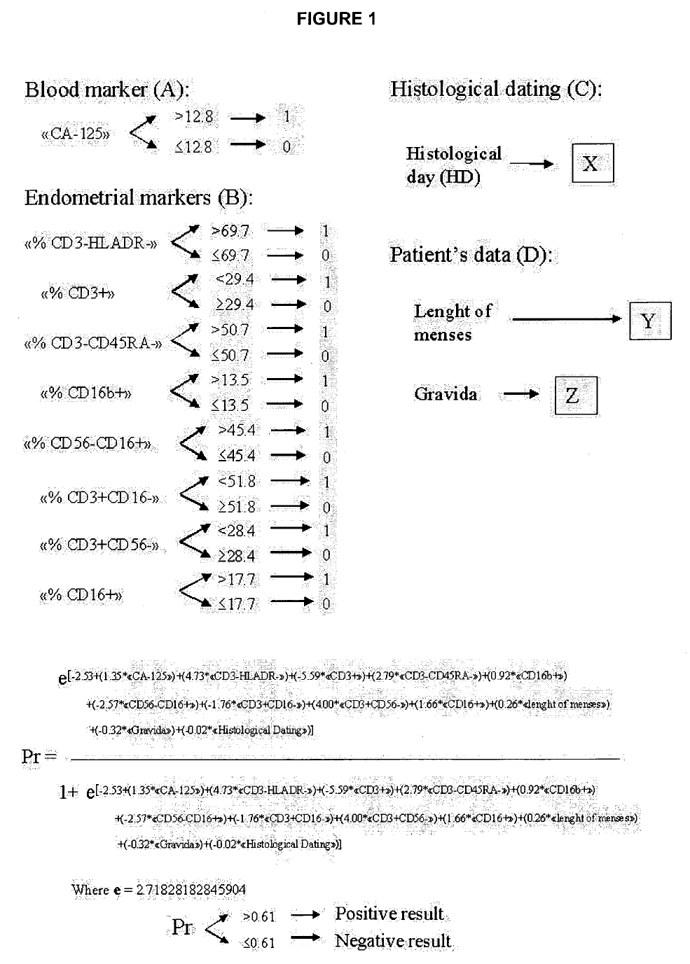

In order to develop the best diagnostic test for endometriosis, these markers were combined together with CD16b+ endometrial leukocytes in a logistic regression model. The quantitative levels of CD3−HLADR−, CD3+, CD3−CD45RA−, CD16b+, CD56−CD16+, CD3+CD16−, CD3+CD56− and CD16+ endometrial leukocyte subsets as well as CA-125 serum level and number of pregnancies was compared to a cut-off point. A score of 1 (or positive result) was given when the quantitative level of a particular marker fulfilled the condition established by the cut-off point (see FIG. 1), whereas a score of 0 (negative result) was given when the quantitative level of the marker did not fulfill the condition of the cut-off point. The score obtained for each marker is included in a logistic regression model as shown in FIG. 1. The length of periods and the number of pregnancies was also included in the logistic regression model as a continuous variable. In addition, the model was adjusted with the histological day of the menstrual cycle at the time of endometrial tissue collection. A probability of suffering from endometriosis (Pr) was calculated by combining all these markers together in the logistic regression model (see FIG. 1). A diagnosis of endometriosis is given, when the probability of suffering from endometriosis calculated by the model (Pr) exceeded the pre-established threshold value. Results presented in Table 3 hereinafter indicate that the use of CD16b+ endometrial leukocyte population in combination with other endometrial leukocyte population, CA-125 serum level and risk factors clearly improves the predictive value for endometriosis. This is shown by area under the ROC curve, levels of sensibility and specificity as well as the positive and negative predictive values.

TABLE 3
Diagnostic performance of CD16b+ endometrial leukocytes when combined to other leukocyte
subsets, CA-125 serum level and risk factors for endometriosis.

| Markers included | Coefficient of | Area under | % | % | Positive | Negative |
|---|---|---|---|---|---|---|
| in the model | regression | ROC | specificity | sensitivity | predictive | predictive |
| (See FIG. 1 for details) | (β) | curve | [95% CI] | [95% CI] | value | value |
| HISTOLOGICAL DATING | 0.263 | 0.835 | 95 | 61 | 91 | 75 |
| LENGHT OF PERIODS | −0.018 | [0.780-0.890] | | | | |
| GRAVIDA | −0.316 | | | | | |
| CD3−HLADR− (>69.7) | 4.730 | | | | | |
| CD3+ (<29.4) | −5.590 | | | | | |
| CD3−CD45RA− (>50.7) | 2.790 | | | | | |
| CD16b+ (>13.5) | 0.920 | | | | | |
| CD56−CD16+ (>45.4) | −2.570 | | | | | |
| CD3+CD16− (<51.8) | −3.997 | | | | | |
| CD3+CD56− (<28.4) | 1.656 | | | | | |
| CD16+ (>17.7) | 1.353 | | | | | |
| CA-125 (>12.8) | −2.531 | | | | | |
| Constant | 2.718 | | | | | |

3) Conclusion

Overall, the results indicate that CD16b+ endometrial leukocyte population can be used as a new diagnostic marker for endometriosis. Furthermore, the combination of CD16b+ endometrial leukocytes with CD3−HLADR−, CD3+, CD3−CD45RA−, CD56−CD16+, CD3+CD16−, CD3+CD56− and CD16+ endometrial leukocyte subsets together with CA-125 serum level and risk factors represent new and improved diagnostic strategy for endometriosis. Indeed, this marker combination allows to detect females with endometriosis with a high specificity, giving rise to a test with high positive predictive value.

Given the high positive predictive value of this combination, the present invention is mostly useful for the identification of patients with high likelihood of suffering from endometriosis. A positive test result would, thus, accelerate the formal diagnosis by surgery and give access to a faster and more appropriate treatment for endometriosis. However, as the marker combination reported herein does not allow to detect all patients with endometriosis, the negative predictive values are not high enough to conclude that the patients does not have endometriosis. A negative test result should, thus, be interpreted as a low likelihood of having endometriosis, but the possibility of endometriosis should not be completely excluded. The marker combination of the present invention may also serve several other different clinical applications including screening, diagnosis, monitoring and prognosis of endometriosis.

4) Remarks

While several embodiments of the invention have been described, it will be understood that the present invention is capable of further modifications, and this application is intended to cover any variations, uses, or adaptations of the invention, following in general the principles of the invention and including such departures from the present disclosure as to come within knowledge or customary practice in the art to which the invention pertains, and as may be applied to the essential features herein before set forth and falling within the scope of the invention.

Although the present invention mostly refers to a definite cell surface molecule (i.e. CD16b) the invention is not restricted to the measure of this sole cell surface molecule. Indeed, a person skilled in the art will easily understand that several cell surface antigens may define the same population of cells. For instance, it may be envisaged that there are molecules other than CD16b that are also specific to the exact same leukocyte population (e.g. different epitopes, isoforms, subunits, chains, glycosylation or phosphorylation forms, allelic variants, members of the same complex, or an antigen with the same distribution). The present invention also encompasses the use of such molecules.

What is claimed is:

1. A method for determining likelihood of endometriosis in a female subject, comprising the steps of:
a) obtaining from said female a sample of uterine endometrial tissues; and
b) measuring in said sample a quantitative level of a population of CD16b+ endometrial leukocytes,
wherein the quantitative level measured in step b) is indicative of an increased likelihood of endometriosis in said female subject as compared to an endometriosis-free female subject.

2. The method of claim 1, comprising a step c) of comparing the quantitative level measured in step b) to a predetermined cutoff value, wherein an increased quantitative level of said population of CD16b+ leukocytes as compared to the cutoff value is indicative of an increased likelihood of endometriosis in said female subject as compared to an endometriosis-free female subject.

3. The method of claim 1, wherein said quantitative level of said population of CD16b+ endometrial leukocytes corresponds to a proportion of a population of eutopic CD16b+ endometrial leukocytes among total endometrial leukocytes of said female subject.

4. The method of claim 3, wherein said proportion of leukocytes is determined by using an antibody specific for said population of CD16b+ leukocytes.

5. The method of claim 2, wherein in step c), said cutoff value is calculated, and wherein it is obtained by using the steps of:
determining a first quantitative level for said population of CD16b+ leukocytes in a positive reference group of female subjects suffering from endometriosis;
determining a second quantitative level for said population of CD16b+ leukocytes in a negative reference group of endometriosis-free female subjects; and
calculating said cutoff value with said first and second quantitative levels.

6. The method of claim 2, further comprising the step of measuring the quantitative level of at least one further population of endometrial leukocytes or at least one population of blood leukocytes.

7. The method of claim 6, wherein the at least one further population of endometrial leukocytes is selected from the group consisting of CD3−HLADR−, CD3+, CD56−CD16+, CD3+CD16−, CD3+CD56−, CD3−CD45RA− and CD16+.

8. The method of claim 1, wherein in step a), the endometrial tissue is obtained during the secretory phase of the menstrual cycle.

9. The method of claim 1, further comprising the steps of obtaining a blood sample from the female subject and measuring in the serum a quantitative level of CA-125.

10. The method of claim 1, further comprising the step of evaluating in said female subject a medical condition selected from the group consisting of the number of pregnancies, the length of menstruation, and the date in the menstrual cycle at which the endometrial tissue is obtained, wherein said medical condition is indicative of a higher likelihood of endometriosis in said female subject as compared to an endometriosis-free female subject.

11. A method for determining likelihood of endometriosis in a female subject, comprising the steps of:
a) evaluating in said female subject a quantitative level of a population of CD16b+ endometrial leukocytes and a quantitative level of at least one further population of endometrial leukocytes, among total endometrial leukocytes;
b) establishing a cutoff value for each quantitative level evaluated in step a);
c comparing each of said quantitative levels evaluated in step a) with the cutoff value defined in step b) and assigning a first value if said quantitative level meets a condition established by the cutoff value, or assigning a second value different from the first value if said quantitative level does not meet the condition established by the cutoff value; and
d) processing the first or second value of step c) in a logistic regression model in order to obtain a score, said score determining the likelihood of endometriosis in said female subject.

12. The method of claim 11, wherein the at least one further population of endometrial leukocytes is selected from the group consisting of CD3−HLADR−, CD3+, CD56−CD16+, CD3+CD16−, CD3+CD56−, CD3−CD45RA− and CD16+.

13. The method of claim 11, further comprising the steps of obtaining a blood sample from the female subject and measuring in the serum a quantitative level of CA-125.

14. The method of claim 11, further comprising the step of evaluating in said female subject a medical condition selected from the group consisting of the number of pregnancies, the length of menstruation, and the date in the menstrual cycle at which the endometrial tissue is obtained, wherein said medical condition is indicative of a higher likelihood of endometriosis in said female subject as compared to an endometriosis-free female subject.

15. A method for determining likelihood of endometriosis in a female subject, comprising the steps of:
a) evaluating in said female subject a quantitative level of a population of CD16b+ endometrial leukocytes and a quantitative level of at least one further population of endometrial leukocytes, among total endometrial leukocytes;
b) establishing a cutoff value for each quantitative level evaluated in step a);
c) comparing each of said quantitative levels evaluated in step a) with the cutoff value defined in step b) and assigning a first value if said quantitative level meets a condition established by the cutoff value, or assigning a second value different from the first value if said quantitative level does not meet the condition established by the cutoff value;
d) adding up the values assigned in step c) to obtain a score;
e) defining a threshold value for the score obtained in step d); and
f) comparing the score obtained in step d) to the threshold value defined in step e);
wherein when said score is higher than said threshold value, there is an indication of an increased or a likelihood of endometriosis in said female subject as compared to an endometriosis-free female subject.

16. The method of claim 15, wherein in step b), said cutoff value is calculated, and in that it is obtained by using the steps of:
determining a first reference quantitative level for said population of CD16b+ endometrial leukocytes and for said at least one specific population of leukocytes in a positive reference group of female subjects suffering from endometriosis;
determining a second reference quantitative level for said population of CD16b+ endometrial leukocytes and for said at least one specific population of leukocytes in a negative reference group of endometriosis-free female subjects; and
calculating said cutoff value with said first and second reference quantitative levels.

17. The method of claim 15, wherein in step e), said threshold value is calculated, and in that it is obtained by using the steps of:
applying steps a) to d) to a positive reference group of female subjects suffering from endometriosis to obtain a first reference score;
applying steps a) to d) to a negative reference group of endometriosis-free female subjects to obtain a second reference score;
calculating said threshold value with said first and second reference score.

18. The method of claim 15, wherein the at least one further population of endometrial leukocytes is selected from the group consisting of CD3−HLADR−, CD3+, CD56−CD16+, CD3+CD16−, CD3+CD56−, CD3−CD45RA− and CD16+.

19. The method of claim 15, further comprising the steps of obtaining a blood sample from the female subject and measuring in the serum a quantitative level of CA-125.

20. The method of claim 15, further comprising the step of evaluating in said female subject a medical condition selected from the group consisting of the number of pregnancies, the length of menstruation, and the date in the menstrual cycle at which the endometrial tissue is obtained, wherein said medical condition is indicative of a higher likelihood of endometriosis in said female subject as compared to an endometriosis-free female subject.

21. A method for determining likelihood of suffering from endometriosis in a female subject, the method comprising the steps of:
a) obtaining uterine from said female a sample of endometrial tissues;
b) determining in said sample a quantitative level of CD16b+, CD3⁻CD45RA⁻, CD3−HLADR−, CD3+, CD56−CD16+, CD3+CD16−, CD3+CD56−, and CD16+ leukocytes;
c) obtaining a blood sample from the female subject;
d) determining in said blood sample a quantitative level of CA-125;
e) evaluating in the female subject a medical condition selected from the group consisting of the number of pregnancies, the length of periods and the day of the menstrual cycle at which the endometrial tissue is sampled;
wherein when combined, the endometrial leukocytes quantitative levels, the CA-125 quantitative level and the medical condition(s) are indicative of a higher or a lower likelihood of endometriosis in the female subject as compared to an endometriosis-free female subject.

22. A diagnostic kit for determining likelihood of endometriosis in a female subject, comprising:
at least one binding agent that specifically binds to a CD16b+ leukocyte; and
a reagent for detecting the binding agent/CD16b+ leukocyte binding complex.

23. The diagnostic kit of claim 22, further comprising at least one element selected from the group consisting of a support for the at least one binding agent, mixing tubes, buffers, enzymes, and instructions for using said kit.

24. The diagnostic kit of claim 22, wherein said at least one binding agent is a CD16b monoclonal or polyclonal antibody.

25. The diagnostic kit of claim 22, further comprising at least another binding agent that specifically binds to another population of leukocytes.

26. The diagnostic kit of claim 25, wherein said another population of leukocytes is selected from the group consisting of CD3−HLADR−, CD3+, CD56−CD16+, CD3+CD16−, CD3+CD56−, CD3−CD45RA− and CD16+.

27. The diagnostic kit of claim 22, comprising at least one further binding agent that specifically binds to CA-125.
